(19) United States
(12) Patent Application Publication
(10) Pub. No.: US 2025/0082197 A1
(43) Pub. Date: Mar. 13, 2025

(54) AR OPTICAL SYSTEM FOR MEASUREMENT OF OCULAR DYSKINESIA WITH QUALITY OF PHOTOGRAPHED IMAGES AND AR APPARATUS INCLUDING THE SAME

(71) Applicant: Korea Photonics Technology Institute, Gwangju (KR)

(72) Inventors: Seon Man KIM, Jeollanam-do (KR); Min Kyu PARK, Gwangju (KR); Sung Joon KIM, Gwangmyeong-si Gyeonggi-do (KR); Kwang Hoon LEE, Anyang-si Gyeonggi-do (KR)

(73) Assignee: Korea Photonics Technology Institute, Gwangju (KR)

(21) Appl. No.: 18/488,099

(22) Filed: Oct. 17, 2023

(30) Foreign Application Priority Data
Sep. 11, 2023 (KR) 10-2023-0120225
Sep. 11, 2023 (KR) 10-2023-0120229

(51) Int. Cl. A61B 3/113; G02B 27/00; G02B 27/01; G02B 27/10; G06T 19/00

(52) U.S. Cl. CPC A61B 3/113; G02B 27/0093; G02B 27/0101; G02B 27/0172; G02B 27/10; G06T 19/006; G02B 2027/0138; G02B 2027/014

(57) ABSTRACT

Disclosed are an AR optical system for measurement of ocular dyskinesia with improved quality of photographed images, and an AR apparatus including the same. According to one aspect of the present disclosure, there are provided an AR optical system for measurement of ocular dyskinesia with improved quality of photographed images by resolving the problem of uneven brightness and darkness occurring in the images due to structural problems, and an AR apparatus including the same.

### CROSS-REFERENCE TO RELATED APPLICATION

This application claims priority to Korean Patent Application No. 10-2023-0120225 filed on Sep. 11, 2023 and Korean Patent Application No. 10-2023-0120229 filed on Sep. 11, 2023, the entire contents of which are herein incorporated by reference.

### BACKGROUND

### 1. Technical Field

The present disclosure relates to an AR optical system capable of precisely measuring ocular dyskinesia by improving the quality of photographed images, and an AR apparatus including the same.

### 2. Related Art

The content described in this section simply provides background information for this embodiment and does not constitute the related art.

Ocular dyskinesia is a general term for diseases in which the gaze directions of both eyes are inconsistent, and it occurs due to various reasons such as dysfunction of extraocular muscles, brain lesions, cranial nerve abnormalities, visual dysfunction (decreased visual acuity, reduced convergence, hyperopia, etc.), thyroid disease, myasthenia gravis, or congenital abnormalities.

When ocular dyskinesia occurs in an adult, diplopia in which objects appear double occurs to cause great difficulties in living. In particular, in the case of central diplopia, it is the standard for determining level 6 visual impairment. If ocular movement abnormalities occur in children, the vision of one eye is not developed well, which may result in amblyopia, a decrease in binocular vision that prevents both eyes from being used at the same time, or a decrease in stereoscopic vision. Therefore, when ocular dyskinesia occurs, it is desirable to diagnose it early and treat diplopia, binocular vision abnormalities, and the like (based on surgical or non-surgical treatment).

According to data from the Health Insurance Review and Assessment Service, it was understood that the number of patients with ocular dyskinesia in Korea was 147,000 in 2010 and has continued to increase, exceeding 180,000 in 2019. In addition, it was shown in 2019 that medical expenses incurred due to ocular dyskinesia exceeded KRW 40.9 billion. It is estimated that the incidence of ocular dyskinesia has increased in children due to decreased vision and increased incidence of refractive errors. In the case of adults, as cerebral ischemia or cerebral infarction increases due to metabolic disease, etc., it is estimated that ocular dyskinesia caused by cranial nerve abnormalities have increased.

Conventionally, the diagnosis of patients with ocular dyskinesia has been mainly made through measurement of the ocular deviation angle. To measure an ocular deviation angle, the patient is asked to look at a target 6 meters or 33 centimeters away, and the re-fixation movement of the eyes is checked by slowly covering both eyes alternately. After that, the measurement of the ocular deviation angle is performed in such a manner of positioning a prism that refracts light in accordance with the direction of eye deviation, covering both eyes alternately, and finding the prism diopter in which the re-fixation movement is eliminated. This method has a disadvantage in that the test results vary greatly depending on the examiner's skill level, as the examiner should directly observe the patient's eye movements and find the moment when the re-fixation movement of the eyes disappears.

Accordingly, the use of virtual reality (VR) devices as medical devices for screening ocular dyskinesia is being considered. It is determined whether an examinee has ocular dyskinesia by tracking eyeball movements using a VR device. However, since the VR device has a structure in which the imaging optical system is disposed in front of the eyes, there is the inconvenience of having to have a closed environment. In addition, since the camera for tracking the eyeballs in the VR device is structurally bound to be disposed below the left and right eyeballs, the lower side image of the eyeball is used to track eyeball movements. Accordingly, a problem arises in which the VR device's examination results (results of tracking the examinee's eyeball movements) become inaccurate.

### SUMMARY

One embodiment of the present disclosure is directed to providing an AR optical system capable of precisely measuring ocular dyskinesia by tracking eyeball movements of an examinee relatively accurately and an AR apparatus including the same.

Further, one embodiment of the present disclosure is directed to providing an AR optical system for measurement of ocular dyskinesia with improved quality of photographed images by resolving the problem of uneven brightness and darkness occurring in the images due to structural problems, and an AR apparatus including the same.

Further, one embodiment of the present disclosure is directed to providing an AR optical system for measuring ocular dyskinesia that minimizes the occurrence of noise that inevitably occurs during the measurement process depending on structural characteristics, and an AR apparatus including the same.

According to one aspect of the present disclosure, there is provided an augmented reality apparatus for examining an examinee's ocular dyskinesia, the augmented reality apparatus including an image output unit that outputs light corresponding to an augmented reality image, a camera photographing the examinee's eyeball by outputting light to the examinee's eyeball and receiving light reflected from the examinee's eyeball, a beam splitter which reflects or transmits light in the visible wavelength band output from the image output unit or entering the examinee's eyeball from the outside, and causes light in the near-infrared wavelength band output from the camera to enter the pupil of the examinee, a dummy optical system which is disposed between the camera and the beam splitter, light sources which are disposed on a surface of the dummy optical system facing the camera to irradiate light toward the beam splitter, a control unit which controls the operation of the image output unit and the camera, and a power supply unit which supplies power so that each configuration in the augmented reality apparatus is capable of being operated.

According to one aspect of the present disclosure, the beam splitter includes a first surface which is disposed in a direction facing the camera and the examinee's eyeball, and reflects light output from the camera to the examinee's eyeball, and a second surface which is disposed in a direction facing the image output unit and the examinee's eyeball, and allows a portion of each of light output from the image output unit and light of the real world entering from the outside to enter the examinee's eyeball.

According to one aspect of the present disclosure, the light sources irradiate light toward the first surface.

According to one aspect of the present disclosure, the light sources irradiate light in the near-infrared wavelength band.

According to one aspect of the present disclosure, the camera outputs or receives light in the near-infrared wavelength band.

According to one aspect of the present disclosure, there is provided an augmented reality apparatus for examining an examinee's ocular dyskinesia, the augmented reality apparatus including an image output unit that outputs light corresponding to an augmented reality image, a camera photographing the examinee's eyeball by outputting light to the examinee's eyeball and receiving light reflected from the examinee's eyeball, a beam splitter which reflects or transmits light in the visible wavelength band output from the image output unit or entering the examinee's eyeball from the outside, and causes light in the near-infrared wavelength band output from the camera to enter the pupil of the examinee, a dummy optical system which is disposed between the camera and the beam splitter, light sources which are disposed on a surface facing the examinee's eyeball of the beam splitter to irradiate light toward the beam splitter, a control unit which controls the operation of the image output unit and the camera, and a power supply unit which supplies power so that each configuration in the augmented reality apparatus is capable of being operated.

According to one aspect of the present disclosure, the augmented reality apparatus further includes a second dummy optical system disposed on a side surface of the beam splitter opposite to where the dummy optical system is disposed.

According to one aspect of the present disclosure, the dummy optical system and the second dummy optical system increase the angular range for examining the examinee's ocular dyskinesia.

According to one aspect of the present disclosure, there is provided an augmented reality apparatus for examining an examinee's ocular dyskinesia, the augmented reality apparatus including an image output unit that outputs light corresponding to an augmented reality image, a camera photographing the examinee's eyeball by outputting light to the examinee's eyeball and receiving light reflected from the examinee's eyeball, a beam splitter which reflects or transmits light in the visible wavelength band output from the image output unit or entering the examinee's eyeball from the outside, and causes light in the near-infrared wavelength band output from the camera to enter the pupil of the examinee, a dummy optical system which is disposed between the camera and the beam splitter, light sources which are disposed on a surface of the dummy optical system facing the camera and disposed biased in a preset direction, and irradiate light toward the beam splitter, a control unit which controls the operation of the image output unit and the camera, and a power supply unit which supplies power so that each configuration in the augmented reality apparatus is capable of being operated.

According to one aspect of the present disclosure, the beam splitter includes a first surface which is disposed in a direction facing the camera and the examinee's eyeball, and reflects light output from the camera to the examinee's eyeball, and a second surface which is disposed in a direction facing the image output unit and the examinee's eyeball, and allows a portion of each of light output from the image output unit and light of the real world entering from the outside to enter the examinee's eyeball.

According to one aspect of the present disclosure, the light source irradiates light toward the first surface.

According to one aspect of the present disclosure, the light source irradiates light in the near-infrared wavelength band.

According to one aspect of the present disclosure, the camera outputs or receives light in the near-infrared wavelength band.

According to one aspect of the present disclosure, there is provided an augmented reality apparatus for examining an examinee's ocular dyskinesia, the augmented reality apparatus including an image output unit that outputs light corresponding to an augmented reality image, a camera photographing the examinee's eyeball by outputting light to the examinee's eyeball and receiving light reflected from the examinee's eyeball, a beam splitter which reflects or transmits light in the visible wavelength band output from the image output unit or entering the examinee's eyeball from the outside, and causes light in the near-infrared wavelength band output from the camera to enter the pupil of the examinee, a dummy optical system which is disposed between the camera and the beam splitter, light sources which are disposed on a surface of the beam splitter facing the examinee's eyeball and disposed biased in a preset direction, and irradiate light toward the beam splitter, a control unit which controls the operation of the image output unit and the camera, and a power supply unit which supplies power so that each configuration in the augmented reality apparatus is capable of being operated.

As described above, according to one aspect of the present disclosure, there is an advantage in that ocular dyskinesia can be accurately measured by relatively accurately tracking the examinee's eyeball movements.

According to one aspect of the present disclosure, there is an advantage in that the quality of photographed images is improved by solving a problem of uneven brightness and darkness occurring in images due to structural problems.

Further, according to one aspect of the present disclosure, there is an advantage in that the accuracy of measurement results can be improved by minimizing the occurrence of noise that inevitably occurs during the measurement process depending on structural characteristics.

### BRIEF DESCRIPTION OF THE DRAWINGS

FIG. 1 is a view showing the configuration of an AR apparatus according to one embodiment of the present disclosure.

FIGS. 2A and 2B are views illustrating one implementation example of an AR apparatus according to one embodiment of the present disclosure.

FIGS. 3A and 3B are views showing each reflective surface of a beam splitter in an optical system according to one embodiment of the present disclosure.

FIGS. 4A, 4B, 4C, and 4D are views showing the wavelength of light diverging from each reflective surface in a beam splitter according to one embodiment of the present disclosure and the proceeding path of light.

FIG. 5 is a view showing the proceeding path of light entering the first reflective surface of the beam splitter in the optical system according to one embodiment of the present disclosure.

FIGS. 6A, 6B, and 6C are views illustrating a process

in which an examinee performs an examination using an AR apparatus according to one embodiment of the present disclosure.

FIGS. 7A and 7B are views illustrating a process in which an examinee performs an examination using an AR apparatus according to another embodiment of the present disclosure.

FIG. 8 is a view illustrating an image photographed using a conventional examination device.

FIG. 9 is a view illustrating an image photographed using an AR apparatus according to another embodiment of the present disclosure.

FIG. 10 is a view showing a side view of a camera and an optical system according to one embodiment of the present disclosure.

FIGS. 11A and 11B are views showing an appearance in which the second reflective surface is recognized by the camera depending on the distance between the camera and the second reflective surface according to one embodiment of the present disclosure.

FIGS. 12A and 12B are views showing images in which an examinee's eyeball is photographed by a conventional AR apparatus and an AR apparatus according to one embodiment of the present disclosure.

### DETAILED DESCRIPTION

Since the present disclosure can make various changes and have various embodiments, specific embodiments will be illustrated in the drawings and described in detail. However, this is not intended to limit the present disclosure to specific embodiments, and should be understood to include all changes, equivalents, and substitutes included in the spirit and technical scope of the present disclosure. While describing each drawing, similar reference numerals are used for similar components.

Terms such as first, second, A, and B may be used in describing various components, but the components should not be limited by the terms. The above terms are used only for the purpose of distinguishing one component from another. For example, a first component may be named a second component, and similarly, the second component may also be named a first component without departing from the scope of the present disclosure. The term “and/or” includes a combination of a plurality of related described items or any one of the plurality of related described items.

When a component is mentioned to be “linked” or “connected” to other component, it may be directly linked to or connected to the other component, but it should be understood that another component may be present in the middle thereof. Meanwhile, when it is mentioned that a component is “directly linked” or “directly connected” to other component, it should be understood that another component is not present in the middle thereof.

The terms used in this application are only used to describe specific embodiments and are not intended to limit the present disclosure. Singular expressions include plural expressions unless the context clearly dictates otherwise. In this application, terms such as “include” or “have” should be understood as not precluding the existence or addition possibility of features, numbers, steps, operations, components, parts, or combinations thereof described in the specification.

Unless otherwise defined, all terms used herein, including technical or scientific terms, have the same meaning as generally understood by a person of ordinary skill in the art to which the present disclosure pertains.

Terms such as those defined in commonly used dictionaries should be interpreted as having a meaning consistent with the meaning in the context of the related technology, and unless explicitly defined in this application, should not be interpreted in an ideal or excessively formal sense.

Additionally, each configuration, process, operation, method, or the like included in each embodiment of the present disclosure may be shared within the scope of not being technically contradictory to each other.

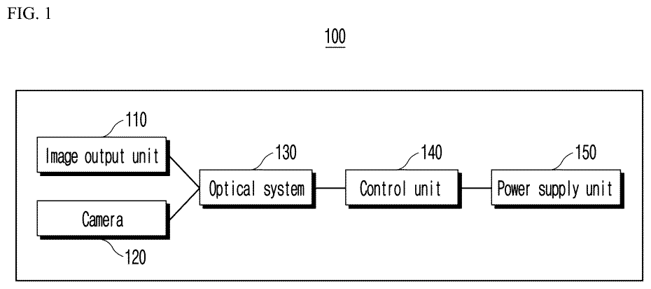

FIG. 1 is a view showing the configuration of an AR apparatus according to one embodiment of the present disclosure.

Referring to FIG. 1, an AR apparatus 100 according to one embodiment of the present disclosure includes an image output unit 110, a camera 120, an optical system 130, a control unit 140, and a power supply unit 150.

The AR apparatus 100 provides augmented reality (AR) images to an examinee and examines the examinee for ocular dyskinesia. The AR apparatus 100 can clearly photograph without error how the examinee's eyeball moves when the AR images are provided by photographing the examinee's eyeball from the front.

Additionally, unintended dark portions may occur in the images photographed depending on the characteristics of the optical configuration included in the optical system. During the process in which the apparatus tracks the pupil's movements, a problem in which dark portions are misrecognized as the pupil may be caused. The AR apparatus 100 minimized the problem in which dark portions are misrecognized as the pupil by reducing the occurrence of dark portions in the photographed images.

The image output unit 110 outputs light corresponding to the augmented reality image. The image output unit 110 receives an augmented reality image generated from the outside and outputs light corresponding thereto. At this time, the image output unit 110 outputs light in the visible light wavelength band.

The camera 120 photographs the examinee's eyeball. The camera 120 photographs the examinee's eyeball by outputting light in the near-infrared wavelength band to the examinee's eyeball and receiving light (in the near-infrared wavelength band) reflected from the examinee's eyeball. At this time, light output from the camera 120 passes through the optical system 130 and can enter/reflect into the center of the examinee's eyeball, thereby allowing the camera 120 to accurately photograph the examinee's eyeball from the front.

The optical system 130 causes light output from the image output unit 110 and light output from the camera 120 to enter the examinee's eyeball, respectively. The optical system 130 allows the examinee to view the augmented reality image by transmitting light output from the image output unit 110 to the examinee. The optical system 130 allows the examinee to recognize the augmented reality image and/or the target for examination by allowing light of the real world together with the augmented reality image to be transmitted to the examinee from the outside. In particular, since the optical system 130 can confirm the target to the examinee in a fairly wide angular range (viewing angle) depending on its structural characteristics, it can improve the accuracy of the examination results. Meanwhile, the optical system 130 similarly causes light output from the camera 120 to enter the examinee's eyeball, and causes light reflected from the examinee's eyeball to advance back to the camera 120. Accordingly, the camera 120 can be allowed to photograph the examinee's eyeball movements.

As described above, the optical system 130 makes each of light output from the image output unit 110 and light output from the camera 120 enter the examinee's eyeball so that there is a possibility that noise may also occur at other portion than the examinee's eyeball when the camera 120 takes pictures of the examinee's eyeball. The optical system 130 may structurally minimize the size of noise occurring in an image photographed by the camera 120. The optical system 130 minimizes inconvenience caused by noise by structurally minimizing the size of noise occurring in the image.

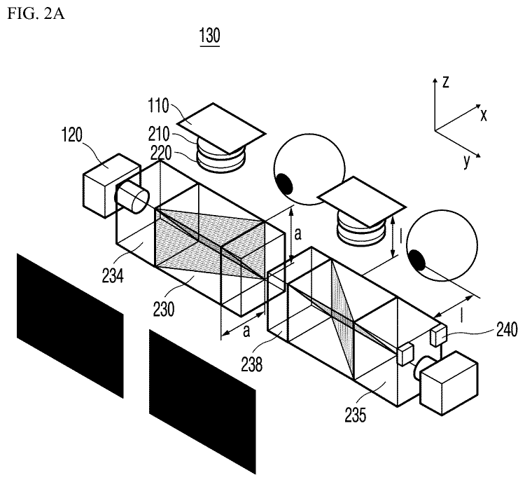

Additionally, the optical system 130 structurally improves the amount of light entering the examinee's eyeball, thereby minimizing shades in the photographed image of the examinee's eyeball. In particular, the pupil recognition rate may be improved by improving the brightness of portions that appear relatively dark in the image according to the structural characteristics of the optical system 130. A more specific description of the optical system 130 will be described later with reference to FIGS. 2 to 12.

The control unit 140 controls the operations of the image output unit 110 and the camera 120. The image photographed by the camera 120 is exported to the outside as it is and may be used to diagnose the examinee's ocular dyskinesia. Alternatively, the control unit 140 may diagnose the examinee's ocular dyskinesia based on the image photographed by the camera 120.

The power supply unit 150 supplies power to respective configurations 110 to 140 to enable each configuration to operate.

FIGS. 2A and 2B are views illustrating one implementation example of an AR apparatus according to one embodiment of the present disclosure.

Referring to FIG. 2A, the optical system 130 according to one embodiment of the present disclosure includes lens units 210 and 220, a beam splitter 230, dummy optical systems 234, and 238, and a light source 240.

The image output unit 110 is disposed vertically above the beam splitter 230 (arranged forward in the eyeball's viewing direction) and outputs an augmented reality image toward the beam splitter 230. As described above, the image output unit 110 outputs an augmented reality image having a visible light wavelength band.

The lens units 210 and 220 concentrate light output from the image output unit 110 to the examinee's pupil. The lens units 210 and 220 are disposed on the path through which the image output unit 110 irradiates light to the beam splitter 230, and concentrate light so that it enters the examinee's pupil in the form of parallel light. Hereby, since the examinee may perceive the augmented reality image as if it is being output from an infinite position, the augmented reality image may have a screen size equivalent to a wide view angle range, and a deep depth and clear image quality characteristics. The lens units 210 and 220 may be implemented with a plurality of spherical lenses as shown in FIGS. 2A and 2B, and may also be implemented with a single or a small number of aspherical lenses.

Meanwhile, the camera 120 is disposed on the side surface (direction perpendicular to the eyeball's viewing direction) that is far from the beam splitter 230 of the dummy optical system 234 so that light for photographing toward the beam splitter 230 via the dummy optical system 234 is output. As described above, the camera 120 outputs light in the near-infrared wavelength band.

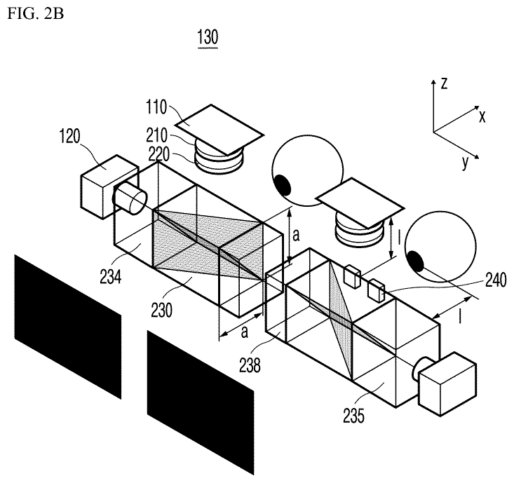

The beam splitter 230 is disposed at an intersection point of the path of light output from the image output unit 110 and the path of light output from the camera 120. The beam splitter 230 reflects or transmits light in the visible light wavelength band that is output from the image output unit 110 or enters the examinee's eyeball from the outside at the corresponding location, and reflects light in the near-infrared wavelength band output from the camera 120 to the examinee's eyeball. The beam splitter 230 includes a plurality of reflective surfaces within one overall configuration, and the respective reflective surfaces are disposed in different directions to reflect entering light in a specific wavelength band in different directions. More specific structures are shown in FIGS. 3A to 5.

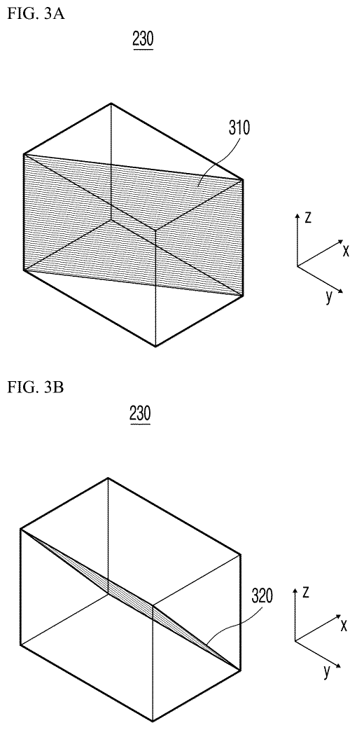

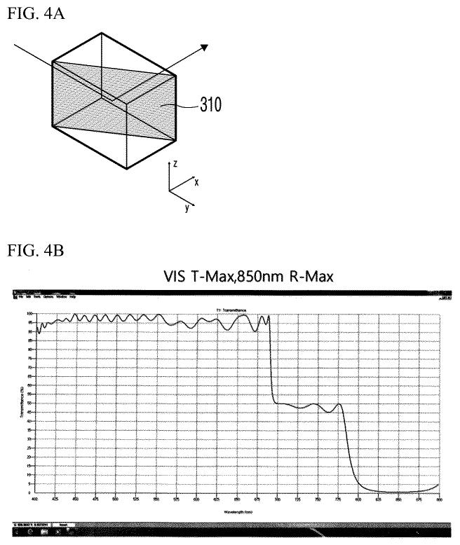

FIGS. 3A and 3B are views showing each reflective surface of a beam splitter in an optical system according to one embodiment of the present disclosure, FIGS. 4A-4D are views showing the wavelength of light diverging from each reflective surface in a beam splitter according to one embodiment of the present disclosure and the proceeding path of light, and FIG. 5 is a view showing the proceeding path of light entering the first reflective surface of the beam splitter in the optical system according to one embodiment of the present disclosure.

Referring to FIGS. 3A, 4A, and 4B, the first surface 310 is disposed in a direction facing the camera 120 and the examinee's eyeball, respectively, and entirely reflects light in the near-infrared wavelength band output from the camera 120 to the examinee's eyeball. As shown in FIG. 4B, the first surface 310 transmits light with respect to most of the visible light wavelength band except for a portion of the red wavelength band without reflection, whereas reflects most of light in the near-infrared wavelength band without transmission.

Meanwhile, referring to FIGS. 3B, 4C, and 4D, the second surface 320 is disposed in a direction facing the image output unit 110 and the examinee's eyeball, respectively. As shown in FIG. 4D, the second surface 320 transmits and reflects light in the visible light wavelength band at a rate of about half, whereas transmits most of light in the near-infrared wavelength band. Accordingly, the second surface 320 transmits a portion of light output from the image output unit 110 as it is without affecting the path of light output from the camera 120, and reflects a portion thereof to the examinee's eyeball. Meanwhile, light of the real world (visible light wavelength band) entering from the outside also passes through the second surface 320, and is partially reflected and partially transmitted to the examinee's eyeball. Accordingly, the examinee may perceive the augmented reality image by receiving the augmented reality image and real world light together.

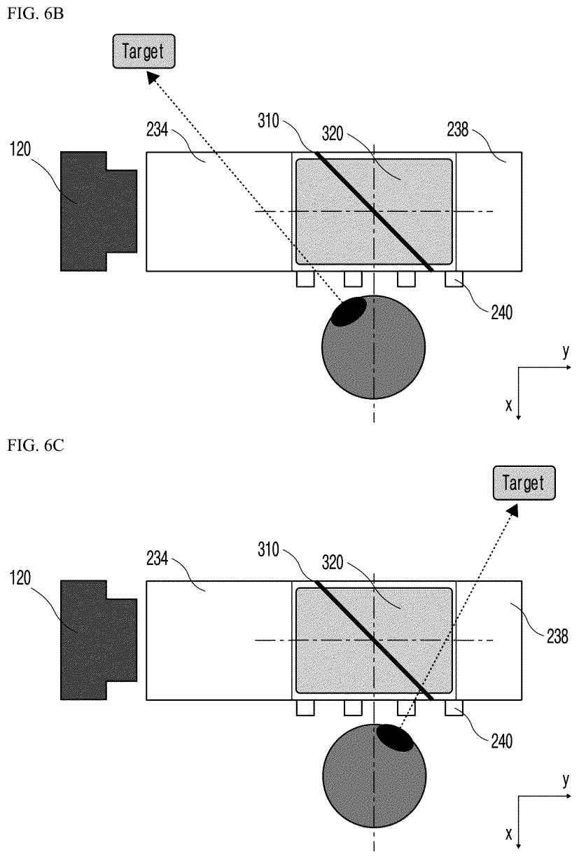

However, since the beam splitter 230 includes a first surface 310 and a second surface 320 disposed in different directions, a phenomenon as shown in FIG. 5 occurs structurally. When light in the near-infrared wavelength band output from the camera 120 enters the first surface 310 and then is reflected, a region that passes through the second surface depending on the region of the first surface 310 may be present, and a region that does not pass through the second surface may be present. The first region 310a in the first surface 310 is a region located above based on the second surface 320 (based on the diagonal line of the first surface) within the first surface 310, and light in the near-infrared wavelength band entering the corresponding region is reflected only through the first surface 310 and proceeds to the examinee's eyeball. Meanwhile, the second region 310b within the first surface 310 is a region located below the second surface 320 (based on the diagonal line of the first surface) within the first surface 310, and light in the near-infrared wavelength band entering the corresponding region is reflected from the first surface 310, additionally passes through the second region 310b, and then proceeds to the examinee's eyeball. Accordingly, light in the near-infrared wavelength band entering the second region 310b passes through the second surface 320, and even if most of light is transmitted, some of it is reflected and light loss may occur. Due to this, the region corresponding to the second region 310b in the image photographed by the camera 120 appears relatively dark compared to the region corresponding to the first region 310a. This can be confirmed in FIG. 8.

FIG. 8 is a view illustrating an image photographed using a conventional examination device.

Referring to FIG. 8, it can be confirmed that the first region 310a is relatively bright based on the second surface 320, whereas the second region 320b is relatively dark. In this way, since the beam splitter 230 includes a first surface 310 and a second surface 320, the image is structurally divided into a relatively dark part and a relatively bright part in the image.

Referring again to FIG. 2A, the beam splitter 230 may allow the camera 120 to photograph the examinee's eyeball from the front using light in the near-infrared wavelength band by having the above-described structure. The AR apparatus 100 may photograph the examinee's eyeball from the front in a point that the camera 120 irradiates light in a path different from that of the image output unit 110, in a point that it irradiates light in different wavelength bands, and in a point that the beam splitter 230 includes the first surface 310 and the second surface 320.

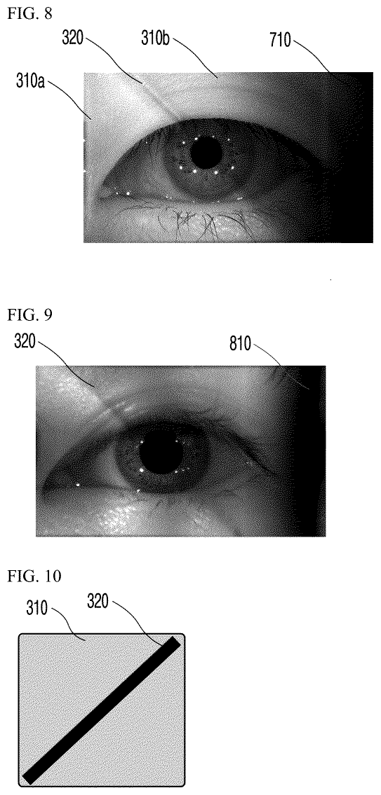

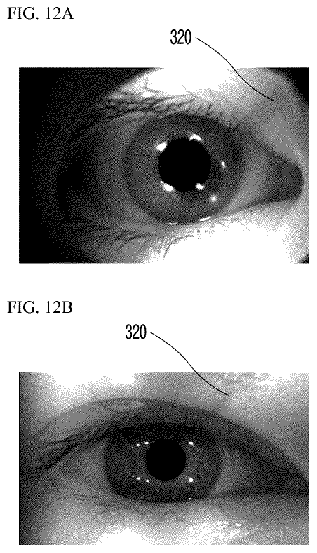

Meanwhile, as the beam splitter 230 has the above-described structure, the side surface portion region of the second surface 320 may occur due to noise in the image photographed by the camera 120. Even if the second surface 320 transmits most of light in the near-infrared wavelength band, a problem may occur in which some of light is reflected from the corresponding surface. This region through which light is reflected like this occurs as noise in the image. The process of generating noise is shown in FIGS. 10 and 12.

FIG. 10 is a view showing a side view of a camera and an optical system according to one embodiment of the present disclosure, and FIGS. 12A and 12B are views showing images in which an examinee's eyeball is photographed by a conventional AR apparatus and an AR apparatus according to one embodiment of the present disclosure.

Referring to FIG. 10, light irradiated from the camera 120 enters the examinee's eyeball by being reflected from the first surface 310 via the dummy optical system 235. At this time, since it does not transmit 100% of light even if the second surface 320 transmits most of light in the near-infrared wavelength band, the second surface 320 is structurally inevitably recognized by the camera 120 as much as the lateral thickness of the second surface 320. That is, the second surface 320 is recognized as shown in FIG. 12A in the image photographed by the camera 120.

Referring to FIG. 12A, it can be confirmed that not only the examinee's eyeball is present in the image photographed by the camera 120, but also noise is generated as much as the lateral thickness of the second surface 320. This corresponds to noise that inevitably occurs optically.

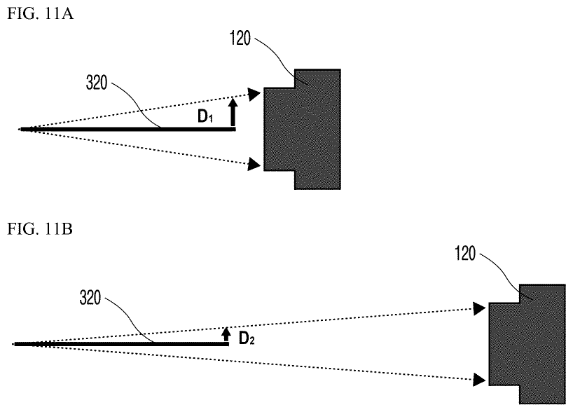

In order to minimize this problem, a dummy optical system 234 is disposed on the side surface of the beam splitter 230 or between the beam splitter 230 and the camera 120. The dummy optical system 234 has the effect of intentionally extending the optical distance between the beam splitter 230 and the camera 120. When the optical distance between the two 120 and 230 increases, the effect shown in FIGS. 11 and 12B may occur.

FIGS. 11A and 11B are views showing an appearance in which the second reflective surface is recognized by the camera depending on the distance between the camera and the second reflective surface according to one embodiment of the present disclosure.

Referring to FIG. 11A, light reflected from the side surface of the second reflective surface 320 enters the camera 120, and the second reflective surface 320 is recognized in the image photographed by the camera 120. At this time, when the dummy optical system 234 does not exist as in the related art so that the distance between the camera 120 and the second reflective surface 320 is relatively close, the second reflective surface 320 is perceived relatively large (D₁) in the image.

Meanwhile, as shown in FIG. 11B, when the distance between the camera 120 and the second reflective surface 320 increases, the second reflective surface 320 is perceived relatively small (D₂) in the image. Accordingly, the second reflective surface 320, which was previously perceived as shown in FIG. 12A in an image photographed by a camera, may be perceived significantly smaller as shown in FIG. 12B.

Referring again to FIG. 2A, as the dummy optical system 234 is disposed in this way, the size of noise generated due to the second reflective surface 320 and all the noise that may be formed in the image photographed by the camera 120 for other reasons may become relatively significantly smaller.

The dummy optical system 234 may have a length (length in the optical axis direction formed by the camera and the beam splitter) that is two times or more the diameter of the lens in the camera 120 in order to reduce the size of noise formed in the image photographed by the camera 120. If the length of the dummy optical system 234 does not satisfy the corresponding length, the effect of reducing the size of noise in the image may deteriorate. In order to prevent this, the dummy optical system 234 may have a preset length (two times or more the diameter of the lens in the camera 120). Accordingly, the AR apparatus 100 may optimally minimize noise occurring in the photographed image.

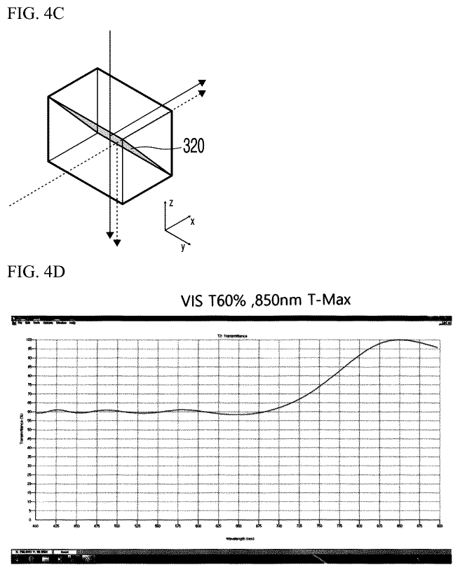

Meanwhile, an additional dummy optical system 238 is disposed on the side surface opposite to the side surface of the beam splitter 230 where the dummy optical system 234 is disposed. As the dummy optical systems 234 and 238 are disposed on both side surfaces of the beam splitter 230, the viewing angle (the angle at which the examinee may look at the target for examination) for examining the examinee's ocular dyskinesia may significantly increase. A more specific description of this will be described later with reference to FIGS. 6A-6C.

The light source 240 is disposed on the surface of the dummy optical system 234 where the camera 120 is disposed, and irradiates light in the near-infrared wavelength band toward the beam splitter 230, more specifically, the first surface 310. Although the camera 120 photographs images in the near-infrared wavelength band, the brightness of the photographed images may be relatively dark since a sufficient amount of light is not provided. To compensate for this, the light source 240 is disposed on the aforementioned surface of the dummy optical system 234 or adjacent to the camera 120 and irradiates light in the same direction as the camera 120. That is, light irradiated from the light source 240 enters the examinee's eyeball, is reflected from the examinee's eyeball, and thus proceeds to the camera 120. Accordingly, the overall brightness of the image in the near-infrared wavelength band photographed by the camera 120 may be brightened.

Meanwhile, referring to FIG. 2B, the optical system 130 according to one embodiment of the present disclosure also includes lens units 210 and 220, a beam splitter 230, dummy optical systems 234 and 238, and a light source 240.

All configurations operate in the same manner, but the light source 240 may be disposed on the surface of the beam splitter 230 that faces the examinee's eyeball, rather than on the surface where the camera 120 of the dummy optical system 234 is disposed. Accordingly, since the light source 240 may irradiate light in the near-infrared wavelength band more directly to the examinee's eyeball, the overall brightness of the image in the near-infrared wavelength band photographed by the camera 120 may be brightened.

FIGS. 6A-6C are views illustrating a process in which an examinee performs an examination using an AR apparatus according to one embodiment of the present disclosure. In FIGS. 6A to 6C, the light source 240 is shown to be disposed on the surface of the beam splitter 230 facing the examinee's eyeball as shown in FIG. 2B, but is not necessarily limited thereto.

Referring to FIG. 6A, light irradiated from the camera 120 is reflected from the first surface 310 via the dummy optical system 234 and enters the examinee's eyeball, and at the same time, light irradiated from the light source 240 also enters the examinee's eyeball. Light that has entered the examinee's eyeball is reflected again from the first surface 310 and enters the camera 120, and the camera may photograph an image of the examinee's eyeball.

At this time, the AR apparatus 100 includes dummy optical systems 234 and 238 on both side surfaces of the beam splitter 230, and thus may enable the examinee to confirm the target in a fairly wide angular range (viewing angle) as shown in FIG. 6B or FIG. 6C. Referring to FIGS. 6B and 6C, in order to check whether the examinee's eye movements are abnormal or not, a target is provided in the region where light from the real world enters the examinee's eyeball via the beam splitter 230. Conventionally, the examinee could check the target only when the path formed between the target and the examinee's eyeball was located in the region inside the beam splitter 230. Because of this, there has been a problem in that the angle range for checking whether the examinee's eye movements are abnormal or not was relatively considerably narrow. Accordingly, it has been difficult for conventional examination devices to sufficiently check whether the examinee's eye movements were abnormal or not. On the other hand, in the AR apparatus 100, the angular range for checking whether the eye movements are abnormal or not can be extended up to the regions of the beam splitter 230 and the dummy optical system 234 and 238. The AR apparatus 100 can check whether the examinee's eye movements are abnormal or not in a sufficiently wide angle range, and thus can relatively accurately check whether the examinee's eye movements are abnormal or not.

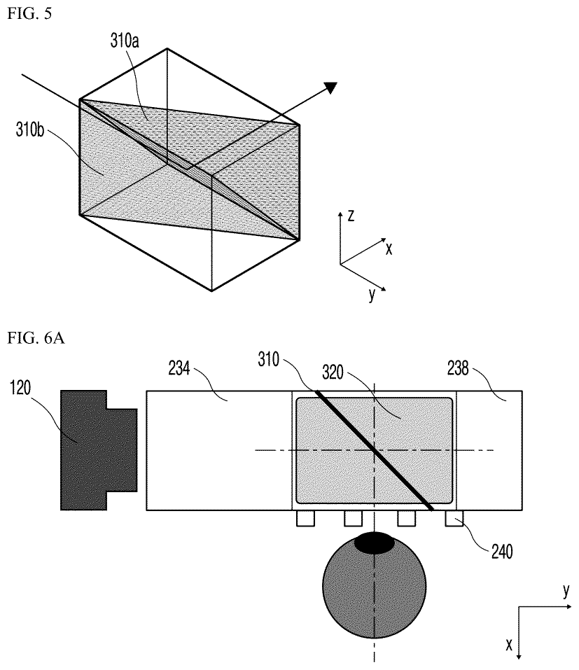

FIGS. 7A and 7B are views illustrating a process in which an examinee performs an examination using an AR apparatus according to another embodiment of the present disclosure. FIG. 7A schematically shows a top view of an AR apparatus according to another embodiment of the present disclosure, and FIG. 7B schematically shows a side view of an AR apparatus according to another embodiment of the present disclosure.

Referring to FIGS. 7A and 7B, when the respective light sources 240 in the AR apparatus 100 according to another embodiment of the present disclosure are disposed on the surface where the camera 120 of the dummy optical system 234 is disposed or the surface of the beam splitter 230 facing the examinee's eyeball, they are not disposed uniformly, but relatively more light sources 240 are disposed biased in a preset direction. Here, the preset direction refers to a direction that satisfies both the lateral direction away from the reference center of the examinee's eyeball and the direction of the second region 310b. Although an ideal beam splitter uniformly reflects light entering at all angles, the beam splitter 230 does not uniformly reflect light entering at all angles in reality, but only completely reflects light entering at a designed angle (for example,) 45°. However, as the angle difference around it increases, the reflectivity of entering light decreases. Due to this, an image such as that in FIG. 8 described above is photographed.

Referring to FIG. 8, in the image of the eyeball photographed by the camera, it can be confirmed that the brightness becomes darker as it goes toward the end based on the center of the eyeball, and a fairly dark region 710 occurs based on the outermost portion of the eyeball. As described above, apart from the fact that relatively dark and bright ones are distinguished depending on the disposition of the first surface 310 and the second surface 320 in the image, the farther the angle of light entering the beam splitter (more specifically, the first surface) is from the center of the examinee's eyeball, the more the dark and bright ones are distinguished. Because of this, the region 710 that simultaneously satisfies the second region 310b and the region located far from the center of the examinee's eyeball has a problem in that it appears relatively significantly darker compared to other regions. In this way, a dark region 710 occurs in the image, and especially when it occurs close to the eyeball, it may cause the following problem. When a conventional examination device uses an eye tracking algorithm in order to determine the movement of the examinee's pupil, a problem may occur in which the eye tracking algorithm misrecognizes not only the examinee's pupil but also the dark region 710 as the pupil.

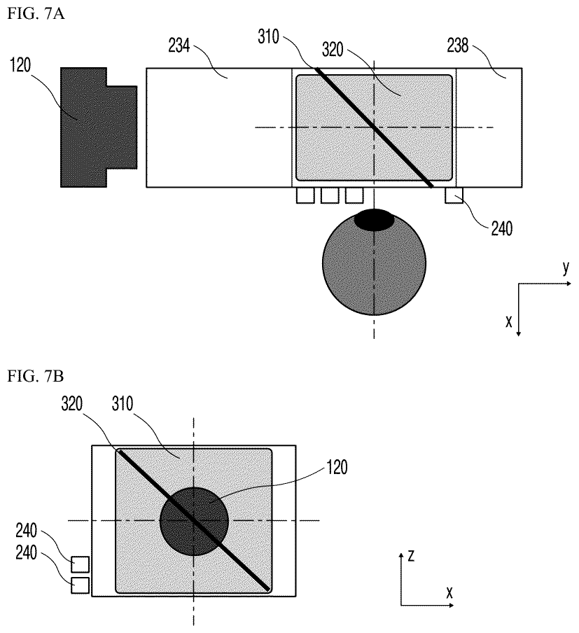

Referring again to FIGS. 7A and 7B, in order to prevent such a problem, the AR apparatus 100 may include light sources 240 disposed biased in a preset direction based on the center of the examinee's eyeball. As described above, due to the characteristics of the beam splitter 230 and the disposition relationship between the first and second surfaces, a region where light enters the examinee's eyeball relatively less is generated. To compensate for such a problem, as the light sources 240 are disposed as described above, more light is incident in a preset direction based on the center of the examinee's eyeball. Accordingly, the camera 120 may acquire an image as shown in FIG. 9.

FIG. 9 is a view illustrating an image photographed using an AR apparatus according to another embodiment of the present disclosure.

Referring to FIG. 9, it can be confirmed that the relatively brightened portion in the second region 320b in the image has increased, and it can be confirmed that the area of the dark region 810 in the image has relatively decreased so that the eyeball is located to be separated from the dark region 810. Due to this, when the AR apparatus 100 tracks the examinee's pupil using an eye tracking algorithm, etc., the problem in which dark portions are misrecognized as the pupil, as in the past, may be minimized.

The above description is merely an illustrative explanation of the technical idea of this embodiment, and those skilled in the art to which this embodiment pertains will be able to make various modifications and variations without departing from the essential characteristics of this embodiment. Accordingly, this embodiment is not intended to limit the technical idea of this embodiment, but rather to explain it, and the scope of the technical idea of this embodiment is not limited by such an embodiment. The scope of protection of this embodiment should be interpreted in accordance with the claims below, and all technical ideas within the scope equivalent thereto should be interpreted as being included in the scope of rights of this embodiment.

This patent is the result of research conducted with the support of the Korea Medical Device Development Fund funded by the Government of the Republic of Korea (Ministry of Science and ICT) in 2023 (Project identification number: 1711179488, detailed project number: 00141436, Project name: Development of an AR technology-based eye movement abnormality screening medical device).

What is claimed is:

1. An augmented reality apparatus for examining an examinee's ocular dyskinesia, the augmented reality apparatus comprising:
an image output unit that outputs light corresponding to an augmented reality image;
a camera photographing the examinee's eyeball by outputting light to the examinee's eyeball and receiving light reflected from the examinee's eyeball;
a beam splitter which reflects or transmits light in a visible wavelength band output from the image output unit or entering the examinee's eyeball from the outside, and causes light in the near-infrared wavelength band output from the camera to enter a pupil of the examinee;
a dummy optical system which is disposed between the camera and the beam splitter to extend an optical distance between the two;
a control unit which controls the operation of the image output unit and the camera; and
a power supply unit which supplies power so that each configuration in the augmented reality apparatus is capable of being operated.

2. The augmented reality apparatus of claim 1, wherein the beam splitter comprises:
a first surface which is disposed in a direction facing the camera and the examinee's eyeball, and reflects light output from the camera to the examinee's eyeball; and
a second surface which is disposed in a direction facing the image output unit and the examinee's eyeball, and allows a portion of each of light output from the image output unit and light of a real world entering from the outside to enter the examinee's eyeball.

3. The augmented reality apparatus of claim 1, wherein the image output unit is disposed vertically above the beam splitter.

4. The augmented reality apparatus of claim 1, wherein the camera is disposed on a side surface far from the beam splitter of the dummy optical system.

5. The augmented reality apparatus of claim 1, wherein the camera outputs or receives light in the near-infrared wavelength band.

6. The augmented reality apparatus of claim 1, wherein the beam splitter is disposed at an intersection point of a path of light output from the image output unit and the path of light output from the camera.

7. The augmented reality apparatus of claim 2, further comprising light sources which are disposed on a surface of the dummy optical system facing the camera or a surface of the beam splitter facing the examinee's eyeball, and irradiate light toward the beam splitter.

8. The augmented reality apparatus of claim 7, wherein the light sources irradiate light toward the first surface.

9. The augmented reality apparatus of claim 7, wherein the light sources are disposed biased in a preset direction.

10. An augmented reality apparatus for examining an examinee's ocular dyskinesia, the augmented reality apparatus comprising:
an image output unit that outputs light corresponding to an augmented reality image;
a camera photographing the examinee's eyeball by outputting light to the examinee's eyeball and receiving light reflected from the examinee's eyeball;
an optical system which reflects or transmits light in a visible wavelength band that is output from the image output unit or entering the examinee's eyeball from the outside, allows light in the near-infrared wavelength band output from the camera to enter into the examinee's pupil, and is capable of structurally minimizing a size of noise occurring in the image photographed by the camera;
a control unit which controls the operation of the image output unit and the camera; and
a power supply unit which supplies power so that each configuration in the augmented reality apparatus is capable of being operated.

11. The augmented reality apparatus of claim 10, wherein the optical system comprises:
a beam splitter which reflects or transmits light in the visible wavelength band that is output from the image output unit or entering the examinee's eyeball from the outside, and allows light in the near-infrared wavelength band output from the camera to enter into the examinee's pupil; and
a dummy optical system which is capable of structurally minimizing the size of noise occurring in the image photographed by the camera.

12. The augmented reality apparatus of claim 11, wherein the dummy optical system is disposed between the camera and the beam splitter to minimize the size of noise occurring in the image photographed by the camera by extending an optical distance between the two.

13. The augmented reality apparatus of claim 10, wherein the image output unit is disposed vertically above a beam splitter.

14. The augmented reality apparatus of claim 10, wherein the camera is disposed on a side surface far from a beam splitter of a dummy optical system.
